(19) United States
(12) Patent Application Publication
(10) Pub. No.: US 2006/0147528 A1
(43) Pub. Date: Jul. 6, 2006

(54) Extended release, multiple unit dosage forms of phenytoin sodium and processes for their preparation

(76) Inventors: Deepak Murpani, Delhi (IN); Rajiv Malik, Wien (AT)

Correspondence Address: RANBAXY INC., 600 COLLEGE ROAD EAST, SUITE 2100, PRINCETON, NJ, 08540, US

(21) Appl. No.: 10/546,113

(22) PCT Filed: Feb. 27, 2004
(86) PCT No.: PCT/IB04/00518

§ 371 Date: Aug. 20, 2005

(30) Foreign Application Priority Data
Feb. 28, 2003 (TN) 204/DE1/2003

(51) Int. Cl. A61K 31/4166; A61K 9/22

(52) U.S. Cl. 424/468; 514/389

(57) ABSTRACT

The present invention relates to oral extended release, multiple unit dosage forms of phenytoin sodium in which individual units comprising phenytoin sodium are coated with one or more film forming polymers. The individual units include between greater than 75% w/w and about 90% w/w of phenytoin sodium.

### TECHNICAL FIELD OF THE INVENTION

The present invention relates to oral extended release, multiple unit dosage forms of phenytoin sodium in which individual units comprising phenytoin sodium are coated with one or more film forming polymers.

### BACKGROUND OF THE INVENTION

Phenytoin is 5,5-diphenyl-2, 4-imidazolidinedione and is a well-known pharmaceutical agent having anti-convulsant and anti-epileptic activity. Due to phenytoin's poor solubility in water, phenytoin sodium, which is much more soluble, is used in the dosage forms.

Phenytoin sodium is available in a number of dosage forms. For example, oral dosage forms include an immediate release capsule, a sustained release capsule (Kapseal), a chewable tablet and an oral suspension. The sustained release capsules are available in two strengths, 30 mg and 100 mg, under the brand name Dilantin®. These capsules contain lactose, confectioner's sugar, talc, magnesium stearate and phenytoin sodium as a loose powder and are band sealed. Drug release problems associated with these pharmaceutical compositions have resulted in numerous recalls because of failure to meet dissolution requirements. Moreover, due to its narrow therapeutic window, it is necessary for patients to take this dosage form several times a day to maintain an effective therapeutic plasma level.

Extended release oral capsules containing 200 mg and 300 mg phenytoin sodium are also commercially available under the brand name Phenytek®. These capsules contain phenytoin sodium in an erodible matrix that includes povidone, hydroxyethyl cellulose, microcrystalline cellulose, magnesium oxide, colloidal silicon dioxide and magnesium stearate, as described in U.S. Pat. No. 6,274,168 and its continuation-in-part application U.S. Patent Application No. 20010043945.

Further, U.S. Pat. No. 5,968,554 discloses a sustained release drug delivery system, which comprises: a core of active ingredient, an enteric coating over the core, a second coating of the active ingredient and finally a coating that is soluble in gastric juices. According to the specifications and examples disclosed in this patent, 305 mg of coated beads were required to deliver a dose of 100 mg phenytoin sodium. Therefore, in order to incorporate a larger dose of the drug, the size of the capsules must be increased, which results in decreasing patient compliance.

U.S. Pat. No. 5,863,558 discloses a dosage form for the controlled release of an antiepileptic drug. The dosage form is characterized by a nonionic polymer film that protects the drug from the fluid of the gastrointestinal environment that contacts the dosage form. The dosage form of this patent includes at least one exit in the inert wall surrounding the internal compartment and the wall maintains its integrity during the release of the drug from the exit.

It is evident that there are a number of extended and sustained release dosage forms available for phenytoin but there is still a need for a dosage form that can incorporate a high dose of phenytoin and impart extended-release properties. Numerous systems have been developed and marketed for the purpose of providing an extended release dosage form and for reducing the number of daily administrations. Examples of such systems are the matrix systems, reservoir systems, osmotic drug delivery systems and other monolithic systems.

For extended-release dosage forms containing very high quantities of the active pharmaceutical ingredient, it is particularly critical to avoid an excessively rapid release (dose dumping) as that can lead to undesirable toxic effects. Moreover, such systems are dependent upon gastric emptying rates and transit times, and can be associated with significant intra- and inter-individual variations.

These disadvantages have led to a shift in modified release technology from the use of monolithic systems to multiple unit systems in which each individual unit is formulated with modified release characteristics. The final dosage form includes a multiplicity of the individual units contained in a formulation in such a form that individual units are made available from the formulation in the gastrointestinal tract.

Multiple unit dosage forms possess a large surface area, which advantageously promotes complete and uniform absorption, minimizes peak plasma fluctuations and thus reduces the potential for systemic side effects. A further advantage of these dosage forms is that high local concentrations of the active substance in the gastrointestinal system is avoided as a consequence of the units being distributed freely throughout the tract. Hence, the multiple unit dosage form ensures incorporation of higher dose of phenytoin, resulting in a decreased dosing frequency and consequently better patient compliance.

### SUMMARY OF THE INVENTION

In one general aspect there is provided an extended-release multiple unit dosage form of phenytoin sodium that includes one or more individual units. The individual units include phenytoin sodium coated with one or more film forming polymers. The individual units include between greater than 75% w/w and up to about 90% w/w of phenytoin sodium.

Embodiments of the extended-release multiple unit dosage form may include one or more of the following features. For example, the dosage form may be one or more of a tablet or a capsule, and, in particular hard gelatin capsules. The dosage form may further include a second amount of phenytoin sodium and, optionally, one or more pharmaceutically inert excipients combined with but separate from the individual units in the multiple unit dosage form. The individual units may include between about 80% w/w and up to about 90% w/w of phenytoin sodium The dosage form may have the following in vitro dissolution profile for phenytoin sodium when tested using USP Apparatus I in water at 50 rpm:
a. not more than 35 percent released in 30 minutes;
b. between 30 and 65 percent released in 60 minutes; and
c. not less than 60 percent released in 120 minutes.

The individual units may be in the form of one or more of pellets, beads, granules, and compacts. The individual units may be prepared by roller compaction, slugging or extrusion-spheronization and, in particular, by roller compaction.

The individual units may further include at least one component selected from the group consisting of lubricants/anti-adherents and glidants. The lubricant/anti-adherent may be selected from one or more of talc, magnesium stearate, calcium stearate, stearic acid, hydrogenated vegetable oil, polyethylene glycol, sodium stearyl fumarate, and sodium benzoate and, in particular, the lubricant/anti-adherent may be magnesium stearate. The glidant may be selected from one or more of colloidal silicon dioxide and talc.

The one or more film forming polymers may be selected from cellulose derivatives, vinyl polymers and copolymers, acrylic polymers and copolymers, and biodegradable polymers. The cellulose derivatives may be selected from carboxymethylcellulose, ethyl cellulose, cellulose acetate, cellulose propionate, cellulose triacetate, cellulose acetate butyrate, hydroxypropyl methylcellulose, hydroxypropyl cellulose, and hydroxyethylcellulose and, in particular, the cellulose derivative may be ethyl cellulose. The vinyl polymers may be selected from polyvinyl pyrrolidone and poly (vinyl) acetate. The acrylic polymers may be selected from cross-linked polyacrylic acids and carbopols. The biodegradable polymers may be selected from polyamino acids and polylactic acid.

The coating may be between about 10% w/w and about 20% w/w of the dosage form. The coating may further include one or more pharmaceutically acceptable excipients selected from plasticizers, lubricants/anti-adherents, and pigments. The plasticizer may be selected from triacetin, triethylcitrate, tributyl citrate, propylene glycol, polyethylene glycol, olive oil, sesame oil, diethyl fumarate and mixtures thereof. The lubricant/anti-adherent may be selected from magnesium stearate, calcium stearate, colloidal silica, hydrogenated vegetable oil, stearic acid, waxes, talc, corn starch, silicon dioxide, sodium lauryl sulfate, and mixtures thereof and, in particular, colloidal silica and/or talc.

In another general aspect there is provided a process for preparing an extended release multiple unit dosage form of phenytoin sodium. The process includes (a) compacting or compressing phenytoin sodium powder to form a compacted material, (b) screening the compacted material to provide uniform sized individual units, and (c) coating the individual units with one or more film-forming polymers. The individual units may include between greater than 75% w/w and up to about 90% w/w of phenytoin sodium

Embodiments of the process may include one or more of the following features. For example, the individual units may include between about 80% and about 90% w/w of phenytoin sodium. The individual units may be in the form of one or more of pellets, beads, granules, and compacts. The individual units may be prepared by roller compaction, slugging or extrusion-spheronization and, in particular, by roller compaction. The process may further include forming one or both of a tablet and a capsule. The process may further include combining the individual units with a second amount of phenytoin sodium and, optionally, one or more pharmaceutically acceptable excipients.

The dosage form may have the following in vitro dissolution profile for phenytoin sodium when tested using USP Apparatus I in water at 50 rpm:
a. not more than 35 percent released in 30 minutes;
b. between 30 and 65 percent released in 60 minutes; and
c. not less than 60 percent released in 120 minutes.

The individual units may further include at least one component selected from the group consisting of lubricants/anti-adherents and glidants. The lubricant/anti-adherent may be selected from talc, magnesium stearate, calcium stearate, stearic acid, hydrogenated vegetable oil, polyethylene glycol, sodium stearyl fumarate and sodium benzoate and, in particular, magnesium stearate. The glidant may be selected from colloidal silicon dioxide and talc.

The one or more film forming polymers may be selected from cellulose derivatives; vinyl polymers and copolymers; acrylic polymers and copolymers; and biodegradable polymers. The cellulose derivatives may be selected from carboxymethylcellulose, ethyl cellulose, cellulose acetate, cellulose propionate, cellulose triacetate, cellulose acetate butyrate, hydroxypropyl methylcellulose, hydroxypropyl cellulose and hydroxyethylcellulose and, in particular, ethyl cellulose. The vinyl polymers may be selected from polyvinyl pyrrolidone and poly (vinyl) acetate. The acrylic polymers may be selected from cross-linked polyacrylic acids and carbopols. The biodegradable polymers may be selected from polyamino acids and polylactic acid.

The coating may be between about 10% w/w and about 20% w/w of the dosage form. The coating may further include one or more pharmaceutically acceptable excipients selected from plasticizers, lubricants/anti-adherents, and pigments. The plasticizer may be selected from triacetin, triethylcitrate, tributyl citrate, propylene glycol, polyethylene glycol, olive oil, sesame oil, diethyl fumarate and mixtures thereof. The lubricant/anti-adherent may be selected from magnesium stearate, calcium stearate, colloidal silica, hydrogenated vegetable oil, stearic acid, waxes, talc, corn starch, silicon dioxide, sodium lauryl sulfate, and mixtures thereof and, in particular, colloidal silica and/or talc.

In another general aspect there is provided a method for one or more of the control of generalized tonic-clonic (grand mal) and complex partial (psychomotor, temporal lobe) seizures and prevention and treatment of seizures occurring during or following neurosurgery. The method includes administering one or more extended-release multiple units of phenytoin sodium coated with one or more film-forming polymers. Each individual unit includes phenytoin sodium and is coated with one or more film-forming polymers. The individual units include between greater than 75% w/w and up to about 90% w/w of phenytoin sodium

Embodiments of the method may include one or more of the following features and those described above. For example, the method may further include a pharmaceutically active agent selected from amongst phenobarbitone and pentobarbital.

The details of one or more embodiments of the invention are set forth in the accompanying description below. Other features and advantages of the invention will be apparent from the description and the claims.

### DETAILED DESCRIPTION

The inventors have developed a dosage form of phenytoin that is capable of incorporating a high dose of the active pharmaceutical ingredient and a dosage form of phenytoin sodium that provides extended release. The extended-release multiple unit dosage forms of phenytoin sodium may be coated with one or more film-forming polymers. The extended release formulation of phenytoin sodium can deliver about 30 mg to about 300 mg of the drug in a single administration without batch-to-batch variation. The extended-release multiple unit dosage forms may further include phenobarbitone and pentobarbital.

The inventors also have developed a process of preparing multiple unit dosage form of phenytoin sodium using dry compaction and applying a coating of film-forming polymers over these multiple units. The extended release multiple unit systems may be formulated by employing a simple process that does not involve the extra steps of layering, drying and band sealing after filing in capsules and nonetheless still may be capable of imparting extended release properties. The process of preparing the oral pharmaceutical extended-release multiple unit dosage form of phenytoin sodium includes: (a) compacting or compressing phenytoin sodium powder, (b) screening the compacted material to form uniform sized multiple units, and (c) coating the individual units with one or more film-forming polymers.

The extended-release multiple unit dosage forms of phenytoin sodium described herein may be used in a method of treatment for the control of generalized tonic-clonic (grand mal) and complex partial (psychomotor, temporal lobe) seizures and for the prevention and treatment of seizures occurring during or following neurosurgery, in a patient in need thereof. The method of treatment includes administering extended-release multiple unit dosage forms of phenytoin sodium coated with one or more film-forming polymers.

The term “extended-release multiple units formulation” indicates a pharmaceutical formulation that includes a multiplicity of individual coated units contained in the formulation in such a form that the individual units will be available from the formulation upon disintegration of the formulation in the stomach. The multiple unit formulation may be a capsule or a tablet that disintegrates in the stomach to give individual units.

Drug release from such extended-release multiple units is controlled either by diffusion, erosion of the coating, and/or by a process dependent on enzymes and/or pH. The erodible coatings involve the use of enteric polymers, which rapidly erode in the intestines.

There are a number of methods available for manufacturing these multiple units. These methods include extrusion-spheronization, wet granulation, and dry granulation. There are two main processes for dry granulation, slugging and roller compaction. Dry granulation or compaction has several advantages because of its low processing time and cost, production of uniform blends and uniform particle size range, improvements in flow properties, reduction in dust, better control of particle hardness and increase in the bulk density of the powder. Dry compaction is an efficient and useful method of granulation that is capable of handling a large amount of material. The compacted granules, being denser than the parent powder, occupy less volume per unit weight. Therefore, a high drug content can be incorporated in the dosage form using this technique.

Phenytoin sodium may constitute up to about 90% w/w of the individual or compacted units. In particular, the individual units may include between about 25% and 90% w/w of phenytoin sodium. Even more particularly, the individual units may include between about 75% and 90% w/w of phenytoin sodium. Most particularly, the individual units may include phenytoin sodium at a value greater than 75% w/w and up to about 90% w/w, such as for example, between 80% w/w and 90% w/w. The dosage form may be one or more of a tablet or a capsule. The multiple units of this invention may be designed as granules, pellets, compacts and beads. Phenytoin sodium may be compressed/compacted alone or with at least one component selected from lubricants/anti-adherents, and glidants.

The coating applied to the multiple units of the present invention includes one or more film forming polymers. The film forming polymers according are substantially water insoluble, or sparingly water-soluble, but permit water diffusion. These polymers may be selected from cellulose derivatives; vinyl polymers and copolymers; acrylic polymers and copolymers; and biodegradable polymers.

Suitable cellulose derivatives may include carboxymethyl cellulose, ethyl cellulose, cellulose acetate, cellulose propionate, cellulose triacetate, cellulose acetate butyrate, cellulose acetate phthalate, hydroxypropyl methyl cellulose, hydroxypropyl cellulose, hydroxyl ethyl cellulose, hydroxyl methyl cellulose and the like. Suitable vinyl polymers and copolymers may be selected from polyvinyl pyrrolidone, polypropylene, poly (vinyl) chloride, poly (vinyl) alcohol, poly (vinyl) acetate and the like. Suitable acrylic polymers and copolymers may be selected from cross-linked polyacrylic acids like carbopols. Suitable biodegradable polymers may be selected from polyamino acids, polylactic acid and copolymers.

The film-forming polymers may be admixed with various excipients, such as plasticizers, lubricants, anti-adherents, and pigments.

Suitable examples of plasticizers include triacetin, glycerine, triethyl citrate, tributyl citrate, polyethylene glycol, propylene glycol, olive oil, sesame, oil, diethyl fumarate and mixtures thereof. Suitable lubricants/anti-adherents include magnesium stearate, calcium stearate, colloidal silica, stearic acid, sodium stearate, hydrogenated vegetable oil, waxes, talc, cornstarch, silicon dioxide, sodium lauryl sulphate and metallic stearates.

The film-forming polymers may be applied as a solution or dispersion in a solvent. The solvent may be selected from water, alcohols such as ethyl alcohol or isopropyl alcohol; ketones such as acetone or ethylmethylketone; halogenated hydrocarbons such as dichloroethane and trichloroethane or mixtures thereof.

Any conventional coating equipment may be employed to facilitate coating, including a centrifugal fluidized bed coating apparatus or a pan coating apparatus. The coating may be applied using a conventional coating pan, a spray coater, rotating perforated pan or an automated system. The coated multiple units may be dried in an oven or in a fluidized bed.

The coating may constitute about 10-20% w/w of the formulation.

The coated multiple units are filled into hard gelatin capsules or compressed into tablets that disintegrate in the stomach to make available a multiplicity of individually coated units.

The extended-release formulation shows the following in vitro dissolution profile for phenytoin sodium in water when tested using USP Apparatus I at 50 rpm:
a. not more than 35 percent released in 30 minutes.
b. between 30 and 65 percent released in 60 minutes
c. not less than 60 percent released in 120 minutes.

The following examples illustrate various aspects of the present invention. These examples are for illustration only and should not be construed as limiting the scope of the invention.

### EXAMPLES 1 and 2

mg/Capsule

| | Ingredients | Example-1 | Example-2 |
|---|---|---|---|
| Core | Phenytoin sodium | 300.0 | 300.0 |
| | Magnesium stearate | — | 5 |
| Coating | Ethyl cellulose | 60 | 60 |
| | Dichloromethane | q.s. | q.s. |
| | Isopropyl alcohol | q.s. | q.s. |
| | Talc | 3 | 3 |

Process:

Phenytoin sodium and magnesium stearate (in case of Example 2) are loaded into a twin shell V-blender and blended. This blend is screened and compacted to form pellets. The compacted pellets are coated with a dispersion of ethyl cellulose. These coated pellets are filled into hard gelatin capsules on automatic capsule filling machines. Table 1 shows the dissolution data of phenytoin sodium 300 mg capsules prepared as per the composition of Example 2.

TABLE 1
Comparative in vitro release of phenytoin sodium extended release
capsules of Example 2 and Phenytek ® capsules (300 mg;
marketed by Mylan) using USP Apparatus I/900 ml water/50 rpm

| | Cumulative Percent of phenytoin sodium released (%) | |
|---|---|---|
| Time (hrs.) | Phenytek ® Capsules | Capsules of Example 2 |
| 0.5 | 14 | 15 |
| 1.0 | 35 | 47 |
| 1.5 | 55 | 52 |
| 2.0 | 69 | 61 |

As can be seen from Table 1, both the Phenytek® capsules and the capsules of Example 2 show the following in vitro dissolution profile for phenytoin sodium in water when tested using USP Apparatus I at 50 rpm:
a. not more than 35 percent released in 30 minutes;
b. between 30 and 65 percent released in 60 minutes; and
c. not less than 60 percent released in 120 minutes.

While several particular forms of the invention have been illustrated and described, it will be apparent that various modifications and combinations of the invention detailed in the text can be made without departing from the spirit and scope of the invention. For example, a dosage form can be prepared that includes phenytoin sodium in the individual units and separately phenytoin sodium optionally with one or more pharmaceutically acceptable excipients. If in a capsule, the separate phenytoin sodium and optional excipients are placed in the capsule with the individual units. The separate phenytoin sodium thus functions in an immediate release capacity. The separate phenytoin sodium and optional excipients can be used in a tablet dosage from in a similar manner and for similar purposes (i.e., to provide an immediate release component of the dosage form). Accordingly, it is not intended that the invention be limited, except as by the appended claims.

1. An extended-release multiple unit dosage form of phenytoin sodium comprising one or more individual units, the individual units comprising phenytoin sodium coated with one or more film forming polymers, wherein the individual units comprise between greater than 75% w/w and up to about 90% w/w of phenytoin sodium.

2. The extended release multiple unit dosage form according to claim 1, wherein the individual units comprise between about 80% w/w and about 90% w/w of phenytoin sodium.

3. The extended release multiple unit dosage form according to claim 1, wherein the individual units are in the form of one or more of pellets, beads, granules, and compacts.

4. The extended release multiple unit dosage form according to claim 1, wherein the individual units are prepared by roller compaction, slugging or extrusion-spheronization.

5. (canceled)

6. The extended release multiple unit dosage form according to claim 1, wherein the individual units further comprise at least one component selected from the group consisting of lubricants/anti-adherents and glidants.

7. (canceled)

8. (canceled)

9. (canceled)

10. The extended release multiple unit dosage form according to claim 1, wherein the one or more film forming polymers are selected from cellulose derivatives, vinyl polymers and copolymers, acrylic polymers and copolymers, and biodegradable polymers.

11. (canceled)

12. (canceled)

13. (canceled)

14. (canceled)

15. (canceled)

16. The extended release multiple unit dosage form according to claim 1, wherein the coating comprises between about 10% w/w and about 20% w/w of the dosage form.

17. The extended release multiple unit dosage form according to claim 1, wherein the coating further comprises one or more pharmaceutically acceptable excipients selected from plasticizers, lubricants/anti-adherents, and pigments.

18. The extended release multiple unit dosage form according to claim 17, wherein the plasticizer is selected from triacetin, triethylcitrate, tributyl citrate, propylene glycol, polyethylene glycol, olive oil, sesame oil, diethyl fumarate and mixtures thereof.

19. The extended release multiple unit dosage form according to claim 17, wherein the lubricant/anti-adherent is selected from magnesium stearate, calcium stearate, colloidal silica, hydrogenated vegetable oil, stearic acid, waxes, talc, corn starch, silicon dioxide, sodium lauryl sulfate, and mixtures thereof.

20. (canceled)

21. (canceled)

22. The extended release multiple unit dosage form according to claim 1, wherein the dosage form comprises one or more of a tablet or a capsule.

23. The extended release multiple unit dosage form according to claim 1, wherein the dosage form further comprises a second amount of phenytoin sodium and, optionally, one or more pharmaceutically inert excipients combined with but separate from the individual units in the multiple unit dosage form.

24. The extended release multiple unit dosage form according to claim 1, wherein the dosage form has the following in vitro dissolution profile for phenytoin sodium when tested using USP Apparatus I in water at 50 rpm:
a. not more than 35 percent released in 30 minutes;
b. between 30 and 65 percent released in 60 minutes; and
c. not less than 60 percent released in 120 minutes.

25. A process for preparing an extended release multiple unit dosage form of phenytoin sodium, the process comprising:
(a) compacting or compressing phenytoin sodium powder to form a compacted material,
(b) screening the compacted material to provide uniform sized individual units, and
(c) coating the individual units with one or more film-forming polymers, wherein the individual units comprise between greater than 75% w/w and up to about 90% w/w of phenytoin sodium.

26. The process according to claim 25, wherein the individual units comprise between about 80% w/w and about 90% w/w of phenytoin sodium.

27. The process according to claim 25, wherein the individual units are in the form of one or more of pellets, beads, granules, and compacts.

28. The process according to claim 25, wherein the individual units are prepared by roller compaction, slugging or extrusion-spheronization.

29. (canceled)

30. The process according to claim 25, wherein the individual units further comprise at least one component selected from the group consisting of lubricants/anti-adherents and glidants.

31. (canceled)

32. (canceled)

33. (canceled)

34. The process according to claim 25, wherein the one or more film forming polymers are selected from cellulose derivatives; vinyl polymers and copolymers; acrylic polymers and copolymers; and biodegradable polymers.

35. (canceled)

36. (canceled)

37. (canceled)

38. (canceled)

39. (canceled)

40. The process according to claim 25, wherein the coating comprises between about 10% w/w and about 20% w/w of the dosage form.

41. The process according to claim 25, wherein the coating further comprises one or more pharmaceutically acceptable excipients selected from plasticizers, lubricants/anti-adherents, and pigments.

42. (canceled)

43. (canceled)

44. (canceled)

45. (canceled)

46. (canceled)

47. The process according to claim 25, wherein the multiple unit dosage form has the following in vitro dissolution profile for phenytoin sodium when tested using USP Apparatus I in water at 50 rpm:
a. not more than 35 percent released in 30 minutes;
b. between 30 and 65 percent released in 60 minutes; and
c. not less than 60 percent released in 120 minutes.

48. The process according to claim 25, further comprising combining the individual units with a second amount of phenytoin sodium and, optionally, one or more pharmaceutically acceptable excipients.

49. A method for one or more of the control of generalized tonic-clonic (grand mal) and complex partial (psychomotor, temporal lobe) seizures and prevention and treatment of seizures occurring during or following neurosurgery, the method comprising administering one or more extended-release multiple units of phenytoin sodium, each individual unit comprising phenytoin sodium and being coated with one or more film-forming polymers, wherein the individual units comprise between greater than 75% w/w and up to about 90% w/w of phenytoin sodium.

50. The method according to claim 49, further comprising a pharmaceutically active agent selected from amongst phenobarbitone and pentobarbital.
